(19) United States
(12) Patent Application Publication
(10) Pub. No.: US 2014/0360910 A1
(43) Pub. Date: Dec. 11, 2014

(54) Individually wrapped cotton balls for sanitary infection control, disposable or refillable cotton ball or gauze dispenser dispensing one-at-a-time the individually wrapped cotton balls or gauze for sanitary skin and wound care, and a disposable or refillable cotton ball dispenser infection control all-in-one sanitary station for first-aid, emergency and critical care situations

(71) Applicants: Christine Renee' Adkins, North Canton, OH (US); Kimbra Jean Austin, North Canton, OH (US)

(72) Inventors: Christine Renee' Adkins, North Canton, OH (US); Kimbra Jean Austin, North Canton, OH (US)

(21) Appl. No.: 13/912,198

(22) Filed: Jun. 7, 2013

(51) Int. Cl. A61B 19/02

(52) U.S. Cl. CPC A61B 19/026
USPC 206/570; 206/525

(57) ABSTRACT

The presented invention consists of an individual 1″ size cotton ball to be individually wrapped one-at-a-time for sanitary infection control, a lightweight durable plastic disposable cotton ball or gauze pad dispenser dispensing one-at-a-time the individually wrapped cotton balls or gauze pads for skin and wound care, and a plastic disposable or refillable cotton ball dispenser infection control all-in-one sanitary station providing first-aid supplies. The invention may help improve in potential life threatening medical situations, wherein having the sanitary first-aid supplies available in one convenient location, aids in the safety and protection of the patient and health care providers. The invention is a simple solution in reducing the risk of infection and aiding in the progress toward the elimination of healthcare-associated infections (HAIs).

### BACKGROUND OF THE INVENTION

1. Field of the Invention

The field of art to which the invention pertains is useful towards skin, wound, and cosmetic care, first-aid and medical care, and more particularly, toward sanitary infection control, reducing the risk of infection and aiding in the progress toward the elimination of healthcare-associated infections (HAIs). The subject matter to the claimed invention is an individually wrapped 1″ size cotton ball for sanitary infection control, a disposable dispenser 2.75″ depth×8″ in diameter, dispensing individually wrapped 1″ size cotton balls or 2″×2″ size gauze pads for sanitary skin and wound care, and a cotton ball dispenser all-in-one station 24″ wide×20″ high×3.5″ deep made of abs plastic construction for medical and first-aid supplies, for attending to wounds and injuries in emergency and critical care situations, providing urgent supplies in one place for easy access.

2. Description of the Related Art Including Information Disclosure Statement

The description of the related art of the invention disclosed here relates in general to a field of sanitary skin and wound care. To my knowledge there is no relevant information in the form of prior art patents or published patent applications of which I am aware of in the specified field. There is only a plastic or plastic like container or bag that contains non-wrapped cotton balls. However, describing to the extent practical the state of no prior art, in analysis and in reference to cotton balls and gauze being applied to the wound on the skin, there are no cotton balls that are individually wrapped for sanitary infection control, there is no device of a cotton ball and gauze dispenser dispensing individually wrapped cotton balls and gauze for sanitary infection control, or a cotton ball or gauze dispenser all-in-one station made of durable abs plastic for medical and first aid supplies, for attending to wounds and injuries in emergency and critical care situations, providing urgent supplies in one place for easy access. Also in analysis, an advantage of a device for an individually wrapped cotton ball dispenser for sanitary infection control, in which cotton balls in bags and containers are not individually wrapped and are stuck to each other, is advantageous in reducing the risk of infection, especially when applied on an open wound. For example, in blood laboratories, while analyzing a sample of blood being drawn, many times the cotton balls, which were left in an open container, were reached in and handled by many different hands. Moreover, when removed from the open container, the cotton balls were stuck to each other causing difficulty in handling, which also required even more unsanitary handling on the remaining cotton balls. The left over cotton balls were then placed back in the already open container. Furthermore, the cotton balls that fell out of the hands of the individual handling them and onto the counter, were then picked up and were placed back in the open container and used for the next individual having blood work. In another analysis, this would also solve the disclosed statements in all nail salons.

Furthermore, information disclosed which are also solved by the invention are 1″ cotton balls that are individually wrapped for infection control and sanitary purposes that are packaged in various quantities and in any size plastic bag and container for more easy access and use. Moreover, a device for a disposable dispenser 2.75″ depth×8″ in diameter containing the individually wrapped 1″ size cotton balls or 2″×2″ size gauze pads, for sanitary infection control also solves the above stated problems, by dispensing one individually wrapped 1″ size cotton ball at a time. Further engaging the dispenser will simplify the desired actions. This is also a great benefit and an advantage in the easy access of an abs plastic construction, 24″ wide×20″ high×3.5″ deep cotton ball dispenser all-in-one station for emergency and critical care situations.

In light of the foregoing discussion, there is a need of a simple solution for preventing and reducing the risk of infection and aiding in the progress toward the elimination of healthcare-associated infections to a wound on the skin and providing easy access and use to providing immediate care for first-aid, in emergency and critical situations.

3. Information Disclosure Statement

That no item of information contained in the information disclosure statement was cited in a communication from a foreign patent office in a counterpart foreign application, and, to the knowledge of the person signing the certification after making reasonable inquiry, no item of information contained in the information disclosure statement was known to any individual designated in §1.56(c) more than three months prior to the filing of the information disclosure statement.

### BRIEF SUMMARY OF THE INVENTION

The subject matter to the claimed invention and an object of the present invention is to provide an individual 1″ size cotton ball to be individually wrapped and sealed one-at-a-time, and to be wrapped with a thin covering on on a strip of light weight paper, and perforated at 2.5″ sections wherein the cotton balls are enclosed and a perforation on the corner for an easy tab opening. The individually wrapped 1″ size cotton balls may be packaged in any size plastic bag or container for sanitary infection control and for reducing the risk of infection and aiding in the progress toward the elimination of healthcare-associated infections (HAIs).

The subject matter of another object of the present invention is a simple cotton ball or gauze pad dispenser 2.75″ depth×8″ in diameter. The disposable dispenser is constructed of a durable lightweight plastic. The dispenser also contains an opening 2,75″×1.25″ dispensing the individually wrapped and perforated 1″ size cotton ball or 2″×2″ size gauze pad roll 2.5″×10.5′, individually wrapped and sealed on light weight paper, for one-at-a-time easy access and use. The opening may contain plastic or steel like teeth where the individual cotton ball or gauze pad can be easily torn on the perforated area. If the dispenser is preferred or deemed as refillable, the dispenser made of a durable lightweight plastic may include a retractable knob and a refillable roll. Included are mounting brackets for wall, cabinet, or counters.

The subject matter of another object of the present invention is a cotton ball dispenser all-in-one station made of abs plastic construction, 24″ wide×20″ high×3.5″ deep for medical and first-aid supplies, for attending to wounds and injuries in emergency and critical care situations, providing urgent supplies in one place for easy access. The station includes the above-mentioned dispenser and provision for other first-aid items such as medical gloves, a box of tissues, band-aids, extra gauze, alcohol packets and swabs, hand sanitizer, medical tape, and anti-bacterial ointment. The station also includes an optional removable door that contains a lock.

To achieve the objectives of the present invention, an embodiment of the individually wrapped 1″ size cotton balls, disposable cotton ball or gauze pad dispenser 2.75″ depth×8″ in diameter, containing 2.75″×1.25″ opening for dispensing, and the cotton ball all-in-one sanitary station made of abs plastic construction, 24″ wide×20″ high×3.5″ deep, is provided.

### BRIEF DESCRIPTION OF THE SEVERAL VIEWS OF THE DRAWING

The preferred embodiments of the invention described below include sixteen figures in conjunction with the appended drawings provided to illustrate and not to limit the invention:

FIG. 1 illustrates an individually wrapped and sealed on light weight paper 1″ size cotton ball with a perforated easy lift corner tab for sanitary infection control for first-aid, skin and wound care; aiding in the progress toward the elimination of healthcare-associated infections (HAIs)

FIG. 2 illustrates an individually wrapped one-at-a-time and sealed on light weight paper, and perforated at 1.25″ sections, containing any quantity 1″ size cotton balls, folded in an accordion style

FIG. 3 illustrates a cotton ball roll 2.5″×10.5, individually wrapped and sealed on light weight paper, and perforated at 1.25″ sections, containing 100 1″ size cotton balls, to be placed inside a disposable or refillable plastic dispenser

FIG. 4 illustrates a plastic bag of individually wrapped and sealed 1″ size cotton balls of any quantity

FIG. 5 illustrates a plastic container of individually wrapped and sealed 1″ size cotton balls of any quantity

FIG. 6 illustrates a bag containing a cotton ball roll, 2.5″×10.5, individually wrapped and sealed on light weight paper, and perforated at 1.25″ sections, containing 100 1″ size cotton balls

FIG. 7 illustrates a plastic bag containing a folded accordion style individually wrapped and sealed 1″ size cotton balls of any quantity

FIG. 8 illustrates a front view of a single cotton ball roll 2.5″×10.5′, individually wrapped and sealed on light weight paper, and perforated at 1.25″ sections, containing 100 1″ size cotton balls

FIG. 9 illustrates a front view of a single gauze pad roll 2.5″×10.5′, individually wrapped and sealed on a strip of light weight paper, and perforated at 2.5″ sections, containing 50 2″×2″ size gauze pads

FIG. 10 illustrates a gauze pad roll 2.5″×10.5′, individually wrapped and sealed on a strip of light weight paper, and perforated at 2.5″ sections, containing 50 2″×2″ size gauze pads, to be placed inside a disposable or refillable plastic dispenser

FIG. 11 illustrates a disposable or refillable plastic cotton ball dispenser 2.75″ depth×8″ in diameter, containing 2.75″×1.25″ opening for dispensing containing plastic or steel like teeth where the individually wrapped and sealed 1″ size cotton ball roll can be easily torn on the perforated area. The device easily dispensing the cotton ball roll 2.5″×10.5′, individually wrapped and sealed on light weight paper, and perforated at 125″ sections, containing 100 1″ size cotton balls for easy access and use, for sanitary infection control for skin and wound care, reducing the risk of infection and aiding in the progress toward the elimination of healthcare-associated infections (HAIs)

FIG. 12 illustrates a disposable or refillable plastic gauze pad dispenser 2.75″ depth×8″ in diameter, containing 2.75″×1.25″ opening for dispensing, containing plastic or steel like teeth where the individually wrapped and sealed 2″×2″ size gauze pad ball roll can be easily torn on the perforated area. The device easily dispensing the gauze pad roll 2.5″×10.5′, individually wrapped and sealed on a strip of light weight paper, and perforated at 2.5″ sections, containing 50 2″×2″ size gauze pads for easy access and use, for sanitary infection control for skin and wound care, reducing the risk of infection and aiding in the progress toward the elimination of healthcare-associated infections (HAIs)

FIGS. 13 and 14 illustrates the inner parts of the disposable cotton ball or gauze dispenser, wherein a spindle like object on the inside is attached to a retractable knob on the outside of the dispenser for retracting the cotton ball or gauze roll

FIG. 15 illustrates a disposable cotton ball or gauze dispenser infection control sanitary all-in-one station made of abs plastic construction, 24″ wide×20″ high×3.5″ deep for medical and first-aid supplies, for attending to wounds and injuries in emergency and critical care situations, providing urgent supplies in one place for easy access. The station includes the above-mentioned dispenser and designated sections for other first-aid items such as medical gloves, a box of tissues, band-aids, extra gauze, alcohol packets and swabs, hand sanitizes, medical tape, and anti-bacterial ointment

FIG. 16 illustrates an optional removable door that contains a lock for the cotton ball dispenser all-in-one sanitary station. Included is the wall mounting brackets for mounting the station

### DETAILED DESCRIPTION OF THE INVENTION

While the preferred embodiments of the invention have been illustrated and described, it will be clear that the invention is not limited to these embodiments only. Numerous modifications, changes, variations, substitutions and equivalents will be apparent to those skilled in the art without parting from the spirit and scope of the invention.

In an embodiment, the field of art to which the invention pertains is useful towards skin, wound, and cosmetic care, first-aid and medical care, and more particularly, toward sanitary infection control, reducing the risk of infection and aiding in the progress toward the elimination of healthcare-associated infections (HAIs). The subject matter to the claimed invention is an individually wrapped and sealed 1″ size cotton ball for sanitary infection control, utilizing a disposable or refillable dispenser 2.75″ depth×8″ in diameter, containing 275″×1.25″ opening for dispensing individually wrapped and sealed 1″ size cotton balls or individually wrapped and sealed 2″×2″ size gauze pads for sanitary skin and wound care, and a cotton ball dispenser sanitary all-in-one station made of abs plastic construction, 24″ wide×20″ high×3.5″ deep for medical and first-aid supplies, for attending to wounds and injuries in emergency and critical care situations, providing urgent supplies in one place for easy access.

A written description of the invention, discovery, and the manner and process of making and using the present invention is provided. Each individually wrapped and sealed 1″ size cotton ball and 2″×2″ size gauze pads covered with a thin layer of plastic contains a perforated lift tab on one corner to be easily opened. The 1″ size cotton balls are individually wrapped and sealed on a strip of lightweight paper, and perforated at 1.25″ sections between each cotton ball. The 2″×2″ size gauze pads are individually wrapped and sealed on a strip of lightweight paper, and perforated at 2.5″ sections between each gauze pad. Moreover, the strip of light weight paper can be folded accordion style on a perforation with a second option of rolling the light weight paper to create a roll of individually wrapped and sealed 1″ size cotton balls or 2″×2″ size gauze pads. The individually wrapped and sealed 1″ size cotton balls may be packaged in various quantities and in any size plastic bags or containers. The individually wrapped and sealed 1″ size cotton balls roll 2.5″×10.5′, individually wrapped and sealed on a strip of light weight paper, and perforated at 1.25″ sections, containing 100, 1″ size cotton balls is to be packaged also in the disposable or refillable cotton ball plastic dispenser 2.75″ depth×8″ in diameter, containing 2.75″×1.25″ opening for dispensing. The individually wrapped and sealed 1″ size cotton balls are useful towards skin, wound, and cosmetic care, first-aid and medical care for sanitary infection control. The same application may be used for the gauze pad roll 2.5″×10.5′, individually wrapped and sealed on a strip of light weight paper, and perforated at 2.5″ sections, containing 50, 2″×2″ size gauze pads, when applicable.

The disposable or refillable cotton ball dispenser is made of lightweight durable plastic, 2.75″ depth×8″ in diameter, containing 2.75″×1.25″ opening for dispensing, containing plastic or steel like teeth where the individually wrapped and sealed 1 size cotton ball or the individually wrapped and sealed 2″×2″ size gauze pad can be easily torn on the perforated area. The dispenser contains the roll of individually wrapped and sealed 1″ size cotton balls or the individually wrapped and sealed 2″×2″ size gauze pads as mentioned above. Each cotton ball dispenser contains a roll of 100 of the individually wrapped and sealed 1″ size cotton balls, and each gauze pad dispenser contains a roll of 50 of the individually wrapped and sealed 2″×2″ size gauze pads, while maintaining sanitary cotton balls and gauze pads, for a more safe application for wounds and skin care, and aiding in reducing the spread of healthcare-associated infection. The disposable or refillable dispenser contains a cotton ball roll 2.5″×10.5′, individually wrapped and sealed on a strip of light weight paper, and perforated at 1.25″ sections, containing 100, 1″ size cotton balls, or gauze pad roll 2.5″×10.5′, individually wrapped and sealed on a strip of light weight paper, and perforated at 2.5″ sections, containing 50 2″×2″ size gauze pads that are rolled on to a retractable spindle into a roll, that is then installed into the dispenser. The dispenser contains a retractable knob. The dispenser also comes with female mounting brackets and screws that attaches to a male connector on the bottom or back of the unit for multiple mounting applications.

The cotton ball all-in-one sanitary station made of abs plastic construction, 24″ wide×20″ high×3.5″ deep is designed to contain the plastic disposable or refillable cotton ball dispenser as mentioned above. The sanitary station also includes a dispenser for hand sanitizer, two durable plastic rectangular insert holders to hold medical glove and tissue boxes, a spindle to hold multiple size rolls of medical tape, a long rectangular shaped horizontal holder to hold antibacterial wipes, and two built-in rectangular shaped boxes to hold band-aides and gauze pads. The station is made of abs plastic construction and mounted to a bracket containing screws for a wall or cabinet mounting. The station also contains a removable door with a lock for securing contents when necessary. The designed goal is to provide a single station to house medical and first-aid supplies in one central location, for attending to wounds and injuries in emergency and critical care situations, providing urgent supplies in one place for quick, easy access and use. Furthermore, to help in reducing the spread of health care-associated infections. The same application may be used for the gauze pad dispenser station when applicable.

In an embodiment, there is no knowledge of any prior art provided. To my knowledge there is no relevant information in the form of prior art patents or published patent applications of which am aware of in the specified field. There is only a plastic or plastic like container or bag that contains non-wrapped cotton balls. However, describing to the extent practical the state of no prior art, in analysis and in reference to cotton balls and gauze pads being applied to a wound on the skin, there are no cotton balls that are individually wrapped for sanitary infection control, there is no device of a cotton ball or gauze pad dispenser dispensing individually wrapped cotton balls or gauze pads for sanitary infection control, and there is no cotton ball or gauze pad dispenser all-in-one station made of abs plastic construction for medical and first-aid supplies, for attending to wounds and injuries in emergency and critical care situations, providing urgent supplies in one place for easy access.

In another embodiment, a simple solution for sanitary infection control and for reducing the risk of infection and aiding in the progress toward the elimination of healthcare-associated infections is provided. Solving a problem for sanitary infection control to a wound on the skin, and solving a problem for easy access and use for immediate care in emergency and critical situations is provided. Information disclosed which are also solved by the invention are cotton balls that are individually wrapped and sealed for infection control and sanitary purposes that are packaged in various quantities and in plastic bags and plastic containers for more easy access and use. Moreover, a device for a disposable cotton ball and gauze pad dispenser containing the individually wrapped cotton balls or gauze pads for sanitary infection control, also solves the above stated problems by dispensing one individually wrapped cotton ball or gauze pad at a time. Further, engaging the dispenser will simplify the desired actions. Furthermore, an advantage is the easy access of a cotton ball dispenser all-in-one sanitary station that includes basic first-aid supplies in one convenient location, for immediate care in critical care situations is provided.

Additionally, the individually wrapped and sealed cotton balls, the disposable cotton ball or gauze pad dispenser, and the cotton ball dispenser all-in-one sanitary station may not be limited to and may include any design, shape, or form which may be made available in multiple sizes, but not limited to and including: travel size, compact, medium, large, or any size to meet consumer needs, and any preferred quantity deemed desirable for market.

Another embodiment, is an arrangement of the description of the views of the drawings. The preferred embodiment of the invention described is a total of sixteen figures in conjunction with the appended description and drawings provided to illustrate and not to limit the invention. All are in accordance with an embodiment of the present invention. All the drawings illustrated, to which the invention pertains is useful towards skin, wound, and cosmetic care, first-aid and medical care, and more particularly, toward sanitary infection control, reducing the risk of infection and aiding in the progress toward the elimination of healthcare-associated infections (HAIs), in which:

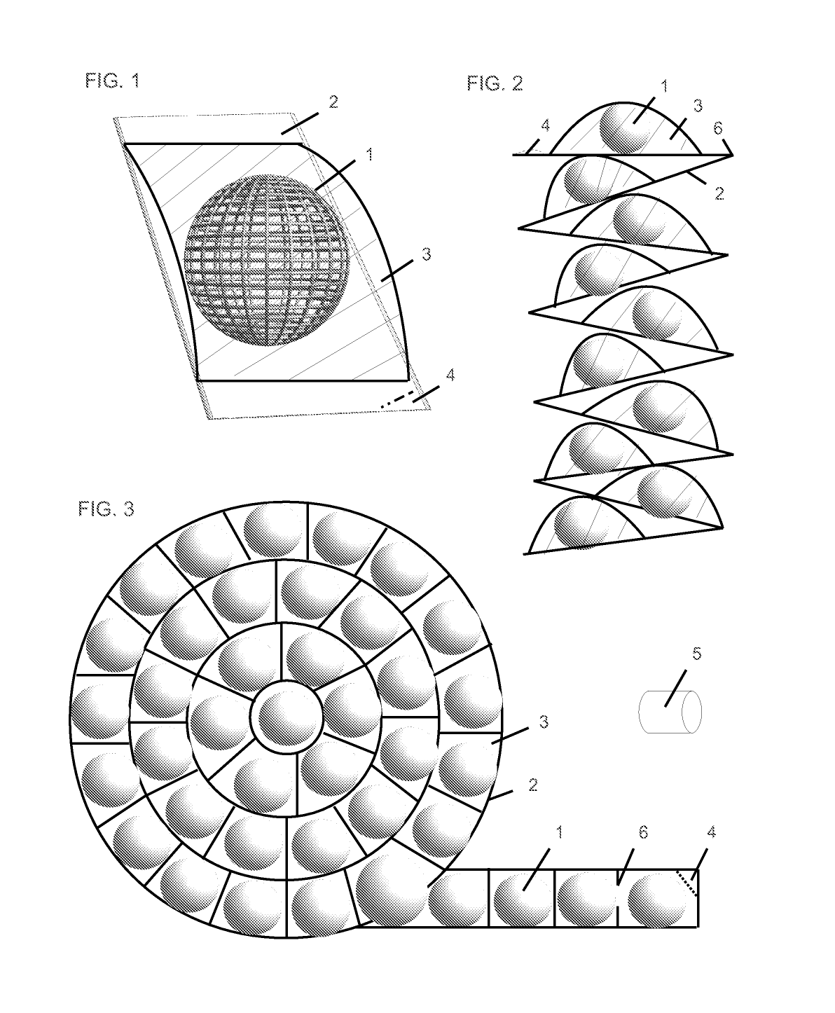

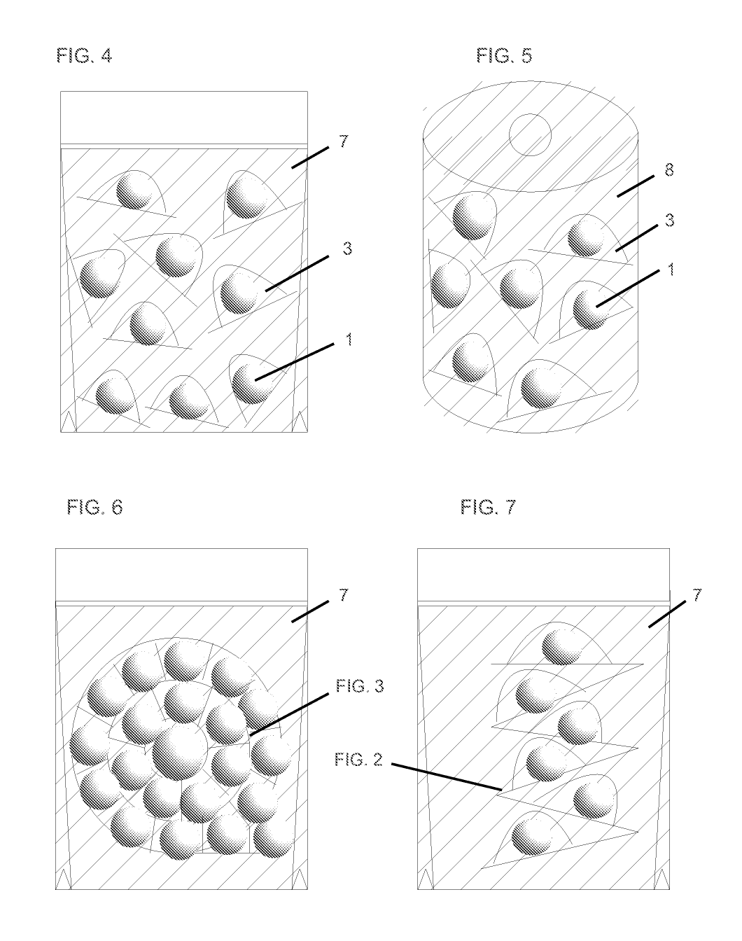

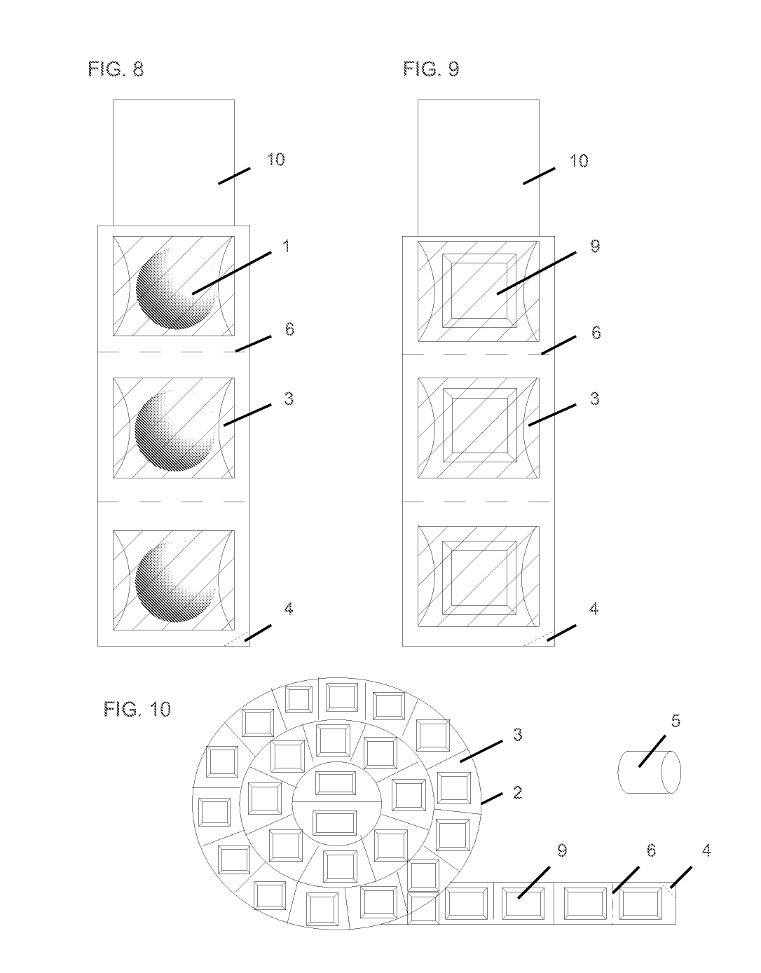

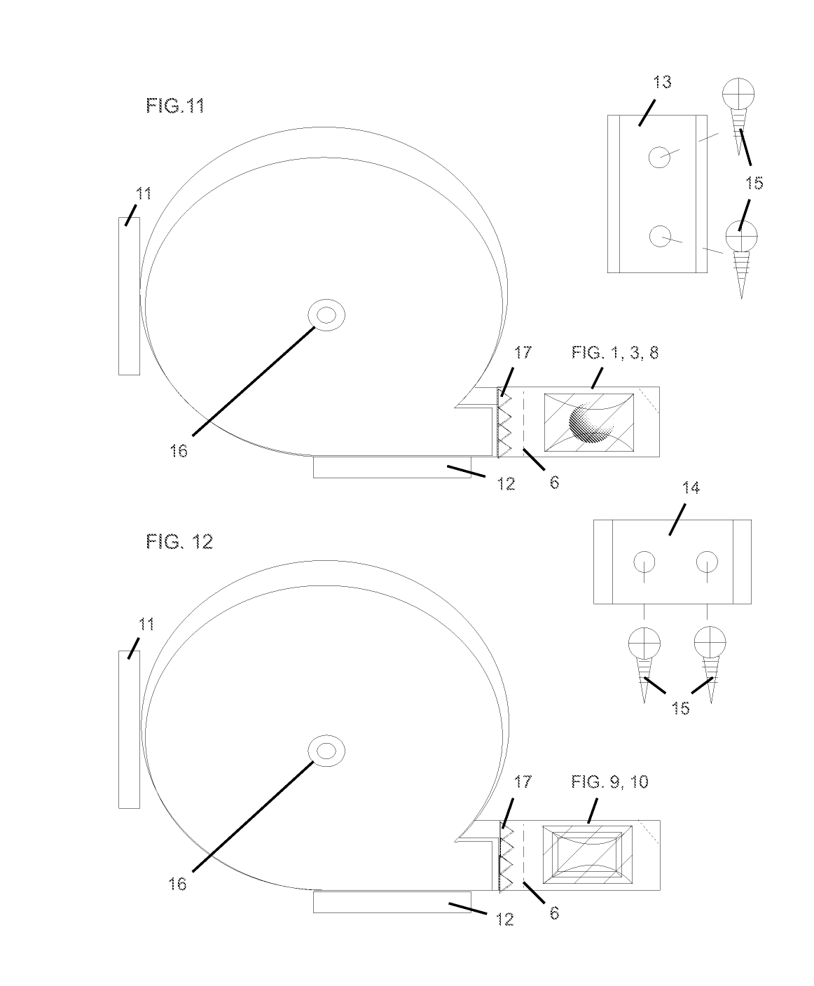

FIG. 1 illustrates a perspective view of an individually wrapped and sealed 1″ size cotton ball. References of the individually wrapped cotton ball include: an individual 1″ size cotton ball 1, light weight paper 2, individually wrapped and sealed 1″ size cotton ball wrap where each cotton ball is covered with a thin layer of plastic or a thin semi transparent paper 3, a perforated lift tab on one corner to be easily opened 4

FIG. 2 illustrates an individually wrapped and sealed and perforated one-at-a-time, 1″ size cotton balls, folded in an accordion style. References here refer to characters of FIG. 1 and include: an individual 1″ size cotton ball 1, light weight paper 2, individually wrapped and sealed 1″ size cotton ball wrap where each cotton ball is covered with a thin layer of plastic or a thin semi transparent paper 3, a perforated lift tab on one corner to be easily opened 4, a perforation on the light weight paper where the accordion style packaging can easily be torn 6

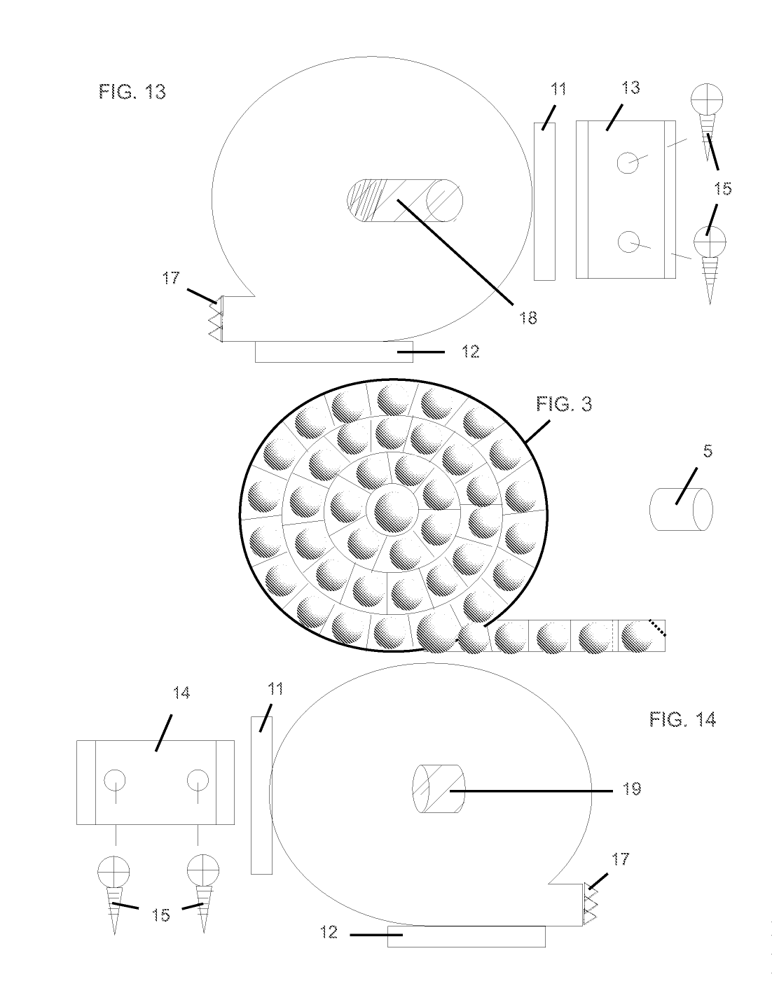

FIG. 3 illustrates a cotton ball roll 2.5″×10.5′, individually wrapped and sealed on a strip of light weight paper, and perforated at 1.25″ sections, containing 100, 1″ size cotton balls, to be placed inside a disposable or refillable plastic dispenser. References to the cotton ball roll include: an individual 1″ size cotton ball 1, light weight paper 2, individually wrapped and sealed 1″ size cotton ball wrap where each cotton ball is covered with a thin layer of plastic or a thin semi transparent paper 3, a perforated if tab on one corner to be easily opened 4, a perforation in the light weight paper where the paper can easily be torn 6 with the plastic or steel like teeth on the opening of the dispenser 17, a spindle for the roll inside the dispenser 5

FIG. 4 illustrates a plastic bag of individually wrapped and sealed 1″ size cotton balls of any quantity. References include: FIG. 1, an individual 1″ size cotton ball 1, an individually wrapped and sealed 1″ size cotton ball wrap where each cotton ball is covered with a thin layer of plastic or a thin semi transparent paper 3, a plastic bag filled with 1″ size cotton balls of any quantity 7 illustrated as FIG. 1

FIG. 5 illustrates a plastic container of individually wrapped and sealed 1″ size cotton balls of any quantity. References include: FIG. 1, an individual 1″ size cotton ball 1, that is a individually wrapped and sealed 1″ size cotton ball wrap where each cotton ball is covered with a thin layer of plastic or a thin semi transparent paper 3

FIG. 6 illustrates a plastic bag containing a cotton ball roll 2.5″×10.5′, individually wrapped and sealed on a strip of light weight paper, and perforated at 1.25″ sections, containing 100, 1″ size cotton balls. References include: FIG. 3 illustrating the cotton ball roll with all references

FIG. 7 illustrates a plastic bag containing folded accordion style individually wrapped and sealed 1″ size cotton balls of any quantity. References include: FIG. 2 with all references of an accordion style packaging, filled into a plastic bag with 1″ size cotton balls of any quantity 7

FIG. 8 illustrates a front view of a cotton ball roll 2.5″×10.5′, individually wrapped and sealed on a strip of light weight paper, and perforated at 1.25″ sections, containing 100 1″ size cotton balls. References include: an individual 1″ size cotton ball 1, an individually wrapped and sealed 1″ size cotton ball wrap where each cotton ball is covered with a thin layer of plastic or a thin semi transparent paper 3, a perforated lift tab on one corner to be easily opened 4, a perforation in the light weight paper 6, adhesive tape at the end of a roll to attach to the spindle in the cotton ball or gauze dispenser 10

FIG. 9 illustrates a front view of a gauze pad roll 2.5″×10.5′, individually wrapped and sealed on a strip of light weight paper, and perforated at 2.5″ sections, containing 50 2″×2″ size gauze pads. References of the gauze pad roll include: an individual 2″×2″ size gauze pad 9, an individually wrapped and sealed 2″×2″ gauze pad wrap where each gauze pad is covered with a thin layer of plastic or a thin semi transparent paper 3, a perforated lift tab on one corner to be easily opened 4, a perforation on the light weight paper 6, adhesive tape at the end of a roll to attach to the spindle in the cotton ball or gauze dispenser 10

FIG. 10 illustrates a gauze pad roll 2.5″×10.5′, individually wrapped and sealed on a strip of light weight paper, and perforated at 2.5″ sections, containing 50, 2″×2″ size gauze pads, to be placed inside a disposable or refillable plastic dispenser. References to the gauze roll include: an individual 2″×2″ size gauze pad 9, light weight paper 2, an individually wrapped and sealed 2″×2″ gauze pad wrap where each gauze pad is covered with a thin layer of plastic or a thin semi transparent paper 3, a perforated lift tab on one corner to be easily opened 4, a perforation on the light weight paper 6, a spindle for the roll inside the dispenser 5

FIG. 11 illustrates a lightweight durable plastic disposable or refillable cotton ball dispenser 2.75″ depth×8″ in diameter, containing 2.75″×1.25″ opening for dispensing containing plastic or steel like teeth where the individually wrapped and sealed 1″ size cotton ball roll can be easily torn on the perforated area. The device easily dispensing the individually wrapped and sealed and perforated one-at-a-time, cotton ball roll for easy access and use, for sanitary infection control for skin and wound care, reducing the risk of infection and aiding in the progress toward the elimination of healthcare-associated infections (HAIs). References to the plastic disposable or refillable cotton ball dispenser include: FIGS. 1, 3, 8 and all of the references to there figures, a perforation on the lightweight paper 6, a male bracket included on the back of the dispenser for mounting 11, a male bracket included on the bottom of the dispenser for mounting 12, a female bracket for the back of the dispenser 13, a female bracket for the bottom of the dispenser 14, screws included for the brackets 15, a retractable knob for the dispenser attached to the inside of the spindle 16, a plastic or steel like teeth at the opening of the dispenser in order to tear on the perforated line of the roll 17

FIG. 12 a lightweight durable plastic disposable or refillable gauze pad dispenser 2.75″ depth×8″ in diameter, containing 2.75″×1.25″ opening for dispensing containing plastic or steel like teeth where the individually wrapped and sealed 2″×2″ size gauze pad roll can be easily torn on the perforated area. The device easily dispensing the individually wrapped and sealed and perforated one-at-a-time, gauze pad roll for easy access and use, for sanitary infection control for skin and wound care, reducing the risk of infection and aiding in the progress toward the elimination of healthcare-associated infections (HAIs). References to the plastic disposable or refillable gauze pad dispenser include: FIGS. 9, 10 and all of the references to there figures, a perforation on the lightweight paper 6, a male bracket included on the back of the dispenser for mounting 11, a male bracket included on the bottom of the dispenser for mounting 12, a female bracket for the back of the dispenser 13, a female bracket for the bottom of the dispenser 14, screws included for the brackets 15, a retractable knob for the dispenser attached to the inside of the spindle 16, a plastic or steel like teeth at the opening of the dispenser in order to tear on the perforated line of the roll 17

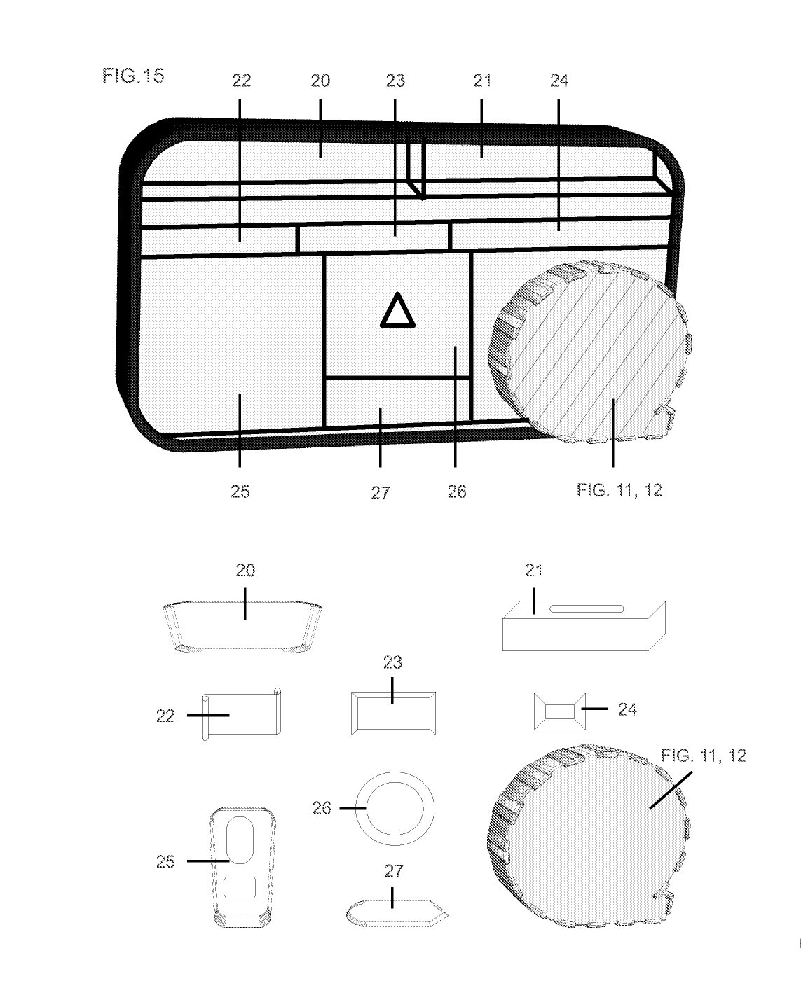

FIGS. 13 and 14 illustrates the inner parts of the plastic disposable or refillable cotton ball or gauze dispenser, wherein a spindle like object on the inside is attached to a retractable knob on the outside of the dispenser for retracting the cotton ball or gauze roll. References include: a male bracket included on the back of the dispenser for mounting 11, a male bracket included on the bottom of the dispenser for mounting 12, a female bracket for the back of the dispenser 13, a female bracket for the bottom of the dispenser 14, screws included for the brackets 15, a plastic or steel like teeth at the opening of the dispenser in order to tear on the perforated line of the roll 17, a spindle object on the inside is attached to a retractable knob on the outside of the dispenser for retracting the cotton ball or gauze roll in or out 18, the spindle connector on the inside of the dispenser 19, a spindle for the roll inside the dispenser 5, and including all the references that go along with FIG. 3 which illustrates an individually wrapped and sealed one-at-a-time and perforated cotton ball roll, to be placed inside the plastic disposable or refillable cotton ball dispenser

FIG. 15 illustrates a plastic disposable cotton ball or gauze pad dispenser infection control sanitary all-in-one station 24″ wide×20″ high×3.5″ deep, made of abs plastic construction, for medical and first-aid supplies, for attending to wounds and injuries in emergency and critical care situations, providing urgent supplies in one place for easy access. The station includes the above-mentioned dispenser and designated sections for other first-aid items such as medical gloves, a box of tissues, band-aids, extra gauze, alcohol packets and swabs, hand sanitizer, medical tape, and anti-bacterial ointment. The references of the cotton ball all-in-one station include: a light weight durable plastic disposable or refillable cotton ball or gauze pad dispenser FIGS. 11 and 12, medical gloves 20, box of tissues 21, band-aids 22, extra gauze pads 23, alcohol packets and swabs 24, hand sanitizer dispenser 25, medical tape 26, anti-bacterial ointment 27, cotton ball station wall mount bracket 30, screws included for the brackets 15

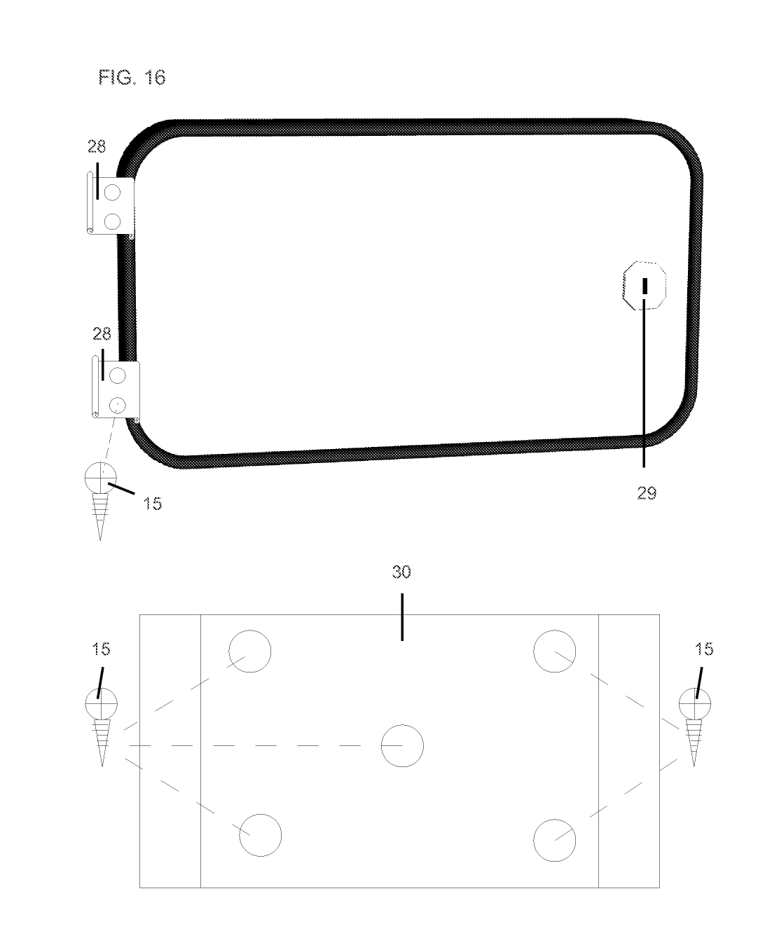

FIG. 16 illustrates an optional removable door that contains a lock for the cotton ball dispenser all-in-one sanitary station. Included is the wall mounting brackets for mounting the station. References to the cotton ball dispenser all-in-one station door includes: hinges for the attachment of the station door 28, a removable door with a lock for securing contents when necessary 29, screws included for the brackets 15, cotton ball station wall mount bracket 30

Furthermore, those with ordinary skill in the art will understand that there may include additional components that are not shown and are not germane to the set up, in accordance with the inventive arrangements and in an embodiment of the present invention.

Additionally, fields of interest for the present invention may include: any medical healthcare facilities, foundations, hospitals, emergency and ambulatory care services and rooms, stat cares, immediate care facilities, doctors offices, Phlebotomists, blood labs, blood drives, blood mobiles, first-aid stations, preventative health care, convalescent centers, school nurse stations, nail salons, spas, animal hospitals, malls, retail stores, and homes.

The invention claimed is:

1. Individually wrapped and sealed 1″ size cotton balls FIG. 1, comprising the elements of an individual 1″ size cotton ball 1, lightweight paper 2, individually wrapped and sealed 1″ size cotton ball wrap where each cotton ball is covered with a thin layer of plastic or a thin semi transparent paper 3, a perforated lift tab on one corner to be easily opened 4, a perforation on the light weight paper where the accordion style packaging can easily be torn 6.

2. A cotton ball roll 2.5″×10,5′, individually wrapped and sealed on a strip of light weight paper, and perforated at 1.25″ sections, containing 100 1 size cotton balls FIG. 3, comprising the elements of an individual 1″ size cotton ball 1, light weight paper 2, individually wrapped and sealed 1″ size cotton ball wrap where each cotton ball is covered with a thin layer of plastic or a thin semi transparent paper 3, a perforated lift tab on one corner to be easily opened 4, a perforation on the lightweight paper where the paper can easily be torn 6.

3. A gauze pad roll 2.5″×10.5′, individually wrapped and sealed on a strip of light weight paper, and perforated at 2.5″ sections, containing 50 2″×2″ size gauze pads FIG. 10, comprising the elements of an individual 2″×2″ size gauze pad 9, light weight paper 2, an individually wrapped and sealed 2″×2″ gauze pad wrap where each gauze pad is covered with a thin layer of plastic or a thin semi transparent paper 3, a perforated lift tab on one corner to be easily opened 4, a perforation on the lightweight paper 6.

4. A lightweight durable plastic disposable or refillable cotton ball dispenser 2.75″ depth×8″ in diameter, containing 2.75″×1.25″ opening for dispensing FIG. 11, FIG. 12, FIG. 13, FIG. 14, comprising the elements of FIGS. 1, 3, 8 and all of the references to there figures, FIGS. 9, 10 and all of the references to there figures, a perforation on the lightweight paper 6, a male bracket included on the back of the dispenser for mounting 11, a male bracket included on the bottom of the dispenser for mounting 12, a female bracket for the back of the dispenser 13, a female bracket for the bottom of the dispenser 14, screws included for the brackets 15, a retractable knob for the dispenser attached to the inside of the spindle 16, a plastic or steel like teeth at the opening of the dispenser in order to tear on the perforated line of the roll 17.

5. A plastic disposable cotton ball or gauze pad dispenser infection control sanitary all-in-one station 24″ wide×20″ high×3.5″ deep, made of abs plastic construction FIG. 15 and FIG. 16, comprising the elements of a lightweight durable plastic disposable or refillable cotton ball or gauze pad dispenser FIG. 11, FIG. 12, medical gloves 20, box of tissues 21, band-aids 22, extra gauze pads 23, alcohol packets and swabs 24, hand sanitizer dispenser 25, medical tape 26, anti-bacterial ointment 27, cotton ball station wall mount bracket 30, screws included for the brackets 15, hinges for the attachment of the station door 28, a removable door with a lock for securing contents when necessary 29.
